(19) United States
(12) Patent Application Publication
(10) Pub. No.: US 2010/0256238 A1
(43) Pub. Date: Oct. 7, 2010

(54) EMULSION-TYPE EXTERNAL PREPARATION, AND METHOD FOR PRODUCTION THEREOF

(75) Inventors: Yasuhisa Tanoue, Kashima-shi (JP); Shigeo Doi, Kashima-shi (JP); Tetsuro Takahashi, Kashima-shi (JP)

Correspondence Address: OBLON, SPIVAK, MCCLELLAND MAIER & NEUSTADT, L.L.P., 1940 DUKE STREET, ALEXANDRIA, VA, 22314, US

(73) Assignee: Yutoku Pharmaceutical Industries Co., Ltd., Kashima-shi (JP)

(21) Appl. No.: 12/714,989

(22) Filed: Mar. 1, 2010

Related U.S. Application Data
(63) Continuation-in-part of application No. PCT/JP2008/065501, filed on Aug. 29, 2008.

(30) Foreign Application Priority Data
Aug. 29, 2007 (JP) 2007-222437

(51) Int. Cl. A61K 31/196; A61K 31/192; A61P 29/00

(52) U.S. Cl. 514/567; 514/570

(57) ABSTRACT

Disclosed are: an emulsion-type external preparation in which the precipitation of crystals of an alkali metal salt or an alkali earth metal salt of a carboxylic acid-type pharmaceutical active ingredient compound or a hydrate of the salt cab be prevented effectively, which is mildly irritative, and in which the decrease in the content of the active ingredient can be prevented; and a method for producing the emulsion-type external preparation.

Specifically disclosed are: an emulsion-type external preparation produced by mixing an emulsion and a treated acidic water-soluble polymer solution, wherein the emulsion is produced by emulsifying the following components (a) and (b): (a) an aqueous phase comprising a pharmaceutical active ingredient compound having a carboxylate group and taking the form of an alkali metal salt or an alkali earth metal salt and an aqueous solvent; and (b) an oily phase comprising at least one oily component selected from the group consisting of a fatty acid ester, hydrocarbon, an animal or plant oil, a hydrogenated oil thereof and a natural wax, and wherein the treated acidic water-soluble polymer solution is prepared by treating the whole or a part of an acidic water-soluble polymer with a basic substance in an aqueous solvent; and a method for producing the emulsion-type external preparation. Also specifically disclosed are: an emulsion-type external preparation produced by mixing an emulsified composition comprising a pharmaceutical active ingredient compound having a carboxylate group and taking the form of an alkali metal salt or an alkali earth metal salt with a solution comprising an acidic water-soluble polymer treated with a basic substance; and a method for producing the emulsion-tube external preparation.

### CROSS-REFERENCE TO RELATED APPLICATIONS

This application is a continuation-in-part of PCT/JP2008/065501, which is hereby incorporated by reference for all purposes.

### TECHNICAL FIELD

This invention relates to an emulsion-type external preparation, and a method for production thereof, and in more detail, to the emulsion-type external preparation and the methods for production thereof which the contents of the active ingredient are not decreased and the active ingredient is not crystallized so that the preparation is superior in the stability, and exhibits low skin-irritation and high safety.

Oral pharmaceutical preparations and parenteral pharmaceutical preparations and the like have been developed as the dosage form of a pharmaceutical preparation to be able to exhibit the appropriate effectiveness in complying with the properties of the active ingredient, the symptoms and age of the patients and the dose and the like. Among the parenteral pharmaceutical preparations, transdermal absorption-type preparations have been developed and widely used to inhibit various unfavorable events such as the occurrence of the side effects by the active ingredient, sudden rise of the plasma concentration immediately after the administration and the like.

Of the transdermal absorption-type preparations, an emulsion-type external preparation such as a cream preparation, a lotion preparation and the like in addition to an oily ointment and gel as the external liniment excels in the compliance to the patients in view of the usability showing the good extensibility when applied to an affected part and the easiness to wash off with water while maintaining the characteristics of the transdermal preparation as mentioned above. From the above reasons, the research and development of the emulsion-type external preparations for the various active ingredients have been progressed.

The external liniments as mentioned above have been developed in the field of pharmaceutical agents inclusive of an anti-inflammatory/analgesic agent, an antifungal agent, an antiitch agent. Especially, in the field of the anti-inflammatory/analgesic agents, gel containing aryl-acetic acid-type diclofenac sodium which exhibits the superior pharmacological effects is put on market. Further, gel containing propionic acid-type loxoprofen sodium which exhibits superior analgesic and anti-inflammatory activities has been investigated.

For example, Patent Literature 1 discloses gel which contains diclofenac sodium as the active ingredient, water, a lower alcohol and a glycol as a solvent, and carboxyvinyl polymer as a gelling agent. Further, Patent Literature 2 discloses a technique to raise a solubility of diclofenac sodium in an oily component by using a fatty acid and a dialkyl ester of carboxylate together. However, the use of the lower alcohol, the fatty acid and the dialkyl ester of carboxylate may bring on skin-irritation.

Further, Patent Literature 3 discloses gel which is intended to enhance the transdermal absorbability by mixing loxoprofen sodium and carboxyvinyl polymer to convert non-free type oily loxoprofen. In Patent Literature 3, sodium ion of loxoprofen sodium was included in carboxyvinyl polymer used as a thickener and gelation was achieved. However, the addition of the lower alcohol or the higher alcohol is essential even in this technique for preparing objective gel and as mentioned above, problems of skin-irritation is caused by the lower alcohol and the contents of loxoprofen as the active ingredient were reduced by forming an ester of loxoprofen in case of the addition of the higher alcohol.

Moreover, in case that the higher alcohol is added to diclofenac sodium for attaining the enhanced transdermal absorbability, unneglectable amounts of ester were formed like loxoprofen as clarified in the experimental examples to be explained laterin the specification by the inventors of the present invention and thus, it is inconvenient to add the higher alcohol to prepare the emulsion-type external preparation.

The external anti-inflammatory/analgesic liniments have been hereinbefore formulated by using the solvents and the solubilizers which are skin-irritant and cause the decrease of the contents of the active ingredient as mentioned above. Consequently, there have been problems that the desired effectiveness has not been demonstrated sufficiently.

The inventors of the present invention have made intensive investigations to develop the emulsion-type external preparations for various pharmaceuticals inclusive of anti-inflammatory/analgesic agents and have attained the knowledge that the desired emulsified composition can not be obtained easily based on the difference of the chemical properties of the active ingredient. Namely, many of the active ingredient compounds of various pharmaceuticals inclusive of anti-inflammatory/analgesic agents have a carboxylic acid group and such compounds having a carboxylic acid group is used in the form of pharmaceutically acceptable salts by converting to an alkali metal salt (sodium salt, potassium salt) or an alkali earth metal salt (calcium salt, magnesium salt). Taking an anti-inflammatory/analgesic agent as an example, by converting an aryl-acetic-type diclofenac and a propionic acid-type loxoprofen to sodium salts thereof, the analgesic and anti-inflammatory activities can be effectively exhibited and such technique have been widely used in clinical fields.

However, these salts of alkali metal and alkali earth metal have problems that they generally form hydrates and are easily crystallized. For example, it is known that loxoprofen is crystallized as a hydrate in the course of the preparation. When mixed with an aqueous solvent, these salts of alkali metal and alkali earth metal have a problem that the crystalline hydrate is easily formed. For example, it is known that diclofenac forms dihydrate or tetrahydrate in an aqueous solvent. Further, while the pharmaceuticals having a carboxylic acid group and taking the form of an alkali metal salt or an alkali earth metal salt inclusive of non-steroidal anti-inflammatory/analgesic agents have ionic properties, the pharmaceuticals themselves have a characteristic that the solubilities to water are not always high and they are easily crystallized when stored at a room temperature for a long time of period or at a low temperature.

As mentioned above, the emulsion-type external preparation comprising the pharmaceutical active ingredient compound having a carboxylic acid group and taking the form of an alkali metal salt or an alkali earth metal salt as an active component were prepared hereinbefore not to be paid attention to their chemical characteristics and to be merely mixed with a solubilizers, thickener and the like. As a result, various problems arose such as the occurrence of skin-irritation by the solubilizers themselves, the decrease of the contents of the active ingredient by forming an ester from the active ingredient compound and a higher alcohol and the like.

Patent Literature 1: Japanese Patent Application Publication (Unexamined) No. Sho. 59-76013 (No. 76013/1984)
Patent Literature 2: Japanese Patent Application Publication (Unexamined) No. Sho. 64-13020 (No. 13020/1989)

### Patent Literature 3: Japanese Patent Application Publication (Unexamined) No. 2001-199883 (No. 199883/2001)

### DISCLOSURE OF INVENTION

### Technical Problem

Therefore, means for inhibiting crystallization effectively, for exhibiting low-irritant property and for preventing the reduction of the contents of the active ingredient have been desired in the preparation of an emulsion-type external preparation containing a pharmaceutical active ingredient compound having a carboxylic acid group and taking the form of an alkali metal salt or an alkali earth metal salt inclusive of a non-steroidal anti-inflammatory/analgesic agent in the form of a hydrate thereof (including a salt formed a hydrate thereof in an aqueous solvent). The technical problem of the present invention is to provide a method for producing such external preparation and the emulsion-type external preparation obtained by such method.

### Technical Solution

The inventors of the present invention have intensively investigated to solve the above-mentioned problems. In consequence, the present inventors had found that an excellent emulsion-type external preparation was obtained by selecting a characteristic formulations and preparing methods for dissolving a salt of an alkali metal or an alkali earth metal of a carboxylic acid-type pharmaceutical active ingredient compound which has a chemical properties to crystallize as a hydrate and to form a hydrate in an aqueous solvent. The excellent emulsion-type external preparation of the present invention does not contain the conventional solvent, does not crystallize for a long time of period, and are not affected by temperature so that it causes low skin-irritation and the decrease of the contents of the active ingredient is not exhibited.

The characteristic formulations and preparing methods as mentioned above are the formulations and the preparing methods to add an acidic water-soluble polymer previously treated by a basic substance to an emulsified composition prepared by emulsifying an aqueous phase containing said active ingredient and an oily phase. Hereby, the inventors of the present invention have found that the crystallization of the active ingredient could be prevented without the use of an irritable solvent such as a lower alcohol or a higher alcohol which is induced an esterification, and have completed this invention.

Namely, the present invention relates to an emulsion-type external preparation prepared by:
emulsifying the following components (a) and (b):
   (a) an aqueous phase containing a pharmaceutical active ingredient compound having a carboxylic acid group and taking a form of an alkali metal salt or an alkali earth metal salt and an aqueous solvent; and
   (b) an oily phase containing one or two or more oily components selected from the group consisting of a fatty acid ester, a hydrocarbon, animal and plant oil and hydrogenated oil thereof and a natural wax; and
mixing the emulsion with a treated acidic water-soluble polymer solution obtained by treating the whole or a part of an acidic water-soluble polymer with a basic substance in an aqueous solvent.

Further, the present invention relates to a method for producing an emulsion-type external preparation prepared by emulsifying the above-mentioned components (a) and (b) and then mixing thus obtained emulsion with a treated acidic water-soluble polymer solution obtainable by treating the whole or a part of an acidic water-soluble polymer with a basic substance in an aqueous solvent.

Furthermore, the present invention relates to an emulsion-type external preparation prepared by preparing an emulsified composition containing a pharmaceutical active ingredient compound having a carboxylic acid group and taking the form of an alkali metal salt or an alkali earth metal salt and mixing the emulsified composition with an acidic water-soluble polymer treated with a basic substance or a solution containing thereof.

Moreover, the present invention relates to a method for producing an emulsion-type external preparation which comprises preparing an emulsified composition containing a pharmaceutical active ingredient compound having a carboxylic acid group and taking the form of an alkali metal salt or an alkali earth metal salt and mixing the emulsified composition with an acidic water-soluble polymer treated with a basic substance.

### EFFECTS OF THE INVENTION

The emulsion-type external preparations of the present invention is highly safe for its low skin-irritant property and highly stable since the contents of the active ingredient are not reduced nor crystallized even when stored for a long time of period. Especially, cream preparation is a useful preparation which excels in the usability with appropriate extensibility and low stickiness in addition to the above-mentioned characteristics.

The active ingredient of the emulsion-type of the present invention is a salt of an alkali metal or an alkali earth metal of a pharmaceutical active ingredient compound having a carboxylic acid group inclusive of non-steroidal anti-inflammatory/analgesic agent (hereinafter referred to as carboxylic acid-type pharmaceutical(s)). These compounds exhibit their specific pharmacological activities by being absorbed through skin or mucous membrane, but these compounds have a tendency to crystallize as a hydrate in a conventional base or to form a hydrate in an aqueous solvent and then crystallize as their chemical characteristics.

As one example of these carboxylic acid-type pharmaceuticals, non-steroidal anti-inflammatory/analgesic agents can be given. The non-steroidal anti-inflammatory/analgesic agents include a phenyl-acetic acid-type or a propionic acid-type anti-inflammatory/analgesic agent. Preferable examples of the phenyl-acetic acid-type anti-inflammatory/analgesic agent include amfenac, felbinac and diclofenac. Among them, diclofenac is particularly preferable. On the other hand, the propionic acid-type anti-inflammatory/analgesic agents include naproxen, pranoprofen, flurbiprofen and loxoprofen. Among them, loxoprofen is particularly preferable.

The salts of alkali metal or alkali earth metal of the carboxylic acid-type pharmaceuticals as mentioned above include, but not to be limited thereto, sodium salts, potassium salts, calcium salts, magnesium salts and the like. Among them, sodium salts and potassium salts are preferable.

As the salts of alkali metal or alkali earth metal of the carboxylic acid-type pharmaceuticals, diclofenac sodium and loxoprofen sodium hydrate which exerts superior anti-inflammatory/analgesic effects are especially preferable.

The examples of the salts of alkali metal or alkali earth metal of the carboxylic acid-type pharmaceuticals which are applicable to the present invention other than non-steroidal anti-inflammatory/analgesic agents include sodium valproate (anti-epileptic agent), anfenac sodium (anti-inflammatory/analgesic agent), indomethacin sodium (anti-inflammatory/analgesic agent), bromfenac sodium (anti-inflammatory/analgesic agent), sodium cromoglicate (anti-allergic agent), sodium canrenoate (diuretic agent), atorbastatin calcium (antilipemic agent), pitavastatin calcium (anti-allergic agent), pravastatin sodium (anti-allergic agent), fluvastatin sodium (anti-allergic agent), rosuvastatin calcium (anti-allergic agent), epoprostenol sodium (prostaglandin I₂ agent), beraprost sodium (prostacyclin (PGI₂) derivative), sodium fusidate (anti-purulent agent), calcium folinate (antidote), mitiglinide calcium (anti-diabetic agent), ozagrel sodium (thromboxane synthetase inhibitor), pemetrexed sodium (metabolic antagonist), talaporfin sodium (anti-tumor agent), porfimer sodium (anti-tumor agent), faropenem sodium (anti-bacterial agent), carumonam sodium (anti-bacterial agent), ampicillin sodium (anti-bacterial agent), oxacillin sodium hydrate (anti-bacterial agent), cloxacillin sodium hydrate (anti-bacterial agent), nafcillin sodium hydrate (anti-bacterial agent), piperacillin sodium (anti-bacterial agent), cefazolin sodium (anti-bacterial agent), cefalotin sodium (anti-bacterial agent), cefodizime sodium (anti-bacterial agent), cefotaxime sodium (anti-bacterial agent), cefoperazone sodium (anti-bacterial agent), cefsulodin sodium (anti-bacterial agent), ceftriaxone sodium (anti-bacterial agent), cefpiramide sodium (anti-bacterial agent), cefbuperazone sodium (anti-bacterial agent), cefminox sodium (anti-bacterial agent), cefmetazole sodium (anti-bacterial agent), flomoxef sodium (anti-bacterial agent), latamoxef sodium (anti-bacterial agent), mupirocin calcium hydrate (anti-bacterial agent) and ceftizoxime sodium (anti-bacterial agent) and the like. The hydrates of the above-mentioned compounds include hemihydrate, monohydrate, sesquihydrate, dihydrate, trihydrate, tetrahydrate and pentahydrate and the like.

The contents of the salts of alkali metal or alkali earth metal of the carboxylic acid-type pharmaceuticals in the emulsion-type external preparations as mentioned above ranges preferably from 0.01 to 10% by weight (hereinafter merely referred to as %), and more preferably from 0.5 to 5%. The case when the contents are less than 0.01% is not preferable because clinically expectable effects can not be desired and the case when the contents are more than 10% is not preferable because the formulations become difficult and the skin-irritation causes frequently.

Further, the aqueous solvents used in the present invention include, for example, water, a glycol such as propylene glycol, 1,3-butylene glycol and polyethylene glycol and a polyvalent alcohol such as glycerin and the like, and one or two or more solvents can be used. Water includes purified water and water for infection and the like. Among them, a mixed solvent of propylene glycol or glycerin and the like with water can be preferably used in view of non-freezing characteristics. On the contrary, a monovalent lower alcohol having one to three carbon atoms is not included in the aqueous solvent of the present invention because of its skin-irritant properties.

The contents of the aqueous solvents in the emulsion-type external preparations preferably ranges from 40 to 90%, more preferably from 50 to 80%. The aqueous solvents are used for dissolving or suspending the salts of alkali metal or alkali earth metal of the carboxylic acid-type compounds which are applicable to the present invention or hydrates thereof (including the hydrate-forming salts in the aqueous solvents; hereinafter defined as same) as mentioned above to prepare the aqueous phase, and further for dissolving or suspending the acidic water-soluble polymer as mentioned later to treat with the basic substance. Furthermore, in case that a part of the acidic water-soluble polymer is treated, the untreated and residual acidic water-soluble polymers are dissolved or suspended and the emulsion is produced by emulsifying the aqueous phase and the oily phase by use thereof.

On the other hand, examples of the oily components include a member selected from the group consisting of a fatty acid ester, a hydrocarbon, an and plant oil and hydrogenated oil thereof and a natural wax.

The above-mentioned ester oils include, for example, hexyl laurate, decyl laurate, isostearyl laurate, isopropyl myristate, butyl myristate, decyl myristate, isotridecyl myristate, myristyl myristate, cetyl myristate, isocetyl myristate, isostearyl myristate, octyldodecyl myristate, isopropyl palmitate, octyl palmitate, cetyl palmitate, isocetyl palmitate, isostearyl palmitate, ethyl stearate, butyl stearate, octyl stearate, isocetyl stearate, stearyl stearate, isopropyl isostearate, hexyl isostearate, isocetyl isostearate, cholesteryl stearate, isostearyl isostearate, octyldecyl isostearate, isoarachyl neopentanoate, octyldodecyl neopentanoate, cetyl 2-ethylhexanoate, stearyl 2-ethylhexanoate, cetosrearyl 2-ethylhexanoate, octyl isononanoate, cetyl caprylate, isostearyl octanoate, hexyldecyl dimethyloctanoate, hexyldecyl neodecanoate, octyldodecyl neodecanoate, diisopropyl adipate, diethyl sebacate, ethyl oleate, decyl oleate, isodecyl oleate, octyldodecyl oleate, ethyl linoleate, isopropyl linoleate, octyldodecyl linoleate, octyldodecyl erucate, cetyl ricinoleate, ethylene glycol dioctanoate, propylene glycol dicaprylate, neopentylglycol dioctanoate, propylene glycol dicaprate, neopentylglycol dicaprate, propylene glycol dicaprylate/dicaprate, ethylene glycol dioleate, trimethylolpropane tri 2-ethylhexanoate, glyceryl tricaprylate/dicaprate, glyceryl tricaprylate, glyceryl triundecanoate, glyceryl triisopalmitate, glyceryl tri isostearate, trimethylolpropane tri isostearate, pentaerythritol tetra 2-ethylhexanoate, cetyl lactate, myristyl lactate and isopropyl lanolate and the like.

Further, the hydrocarbon oils include, for example, squalane, liquid paraffin, α-olefin oligomer, isoparaffin, polyethylene powder, seresin, paraffin, liquid isoparaffin, polybutene, microcrystalline wax, petrolatum, white petrolatum and the like.

Furthermore, the an or plant oils and hydrogenated oils thereof include, for example, animal oils and hydrogenated oils thereof such as tallow, hydrogenated tallow, lard, hydrogenated lard, horse oil, hydrogenated horse oil, mink oil, orange roughy oil, fish oil, hydrogenated fish oil and egg yolk oil and the like and plant oils and hydrogenated oils thereof such as avocado oil, almond oil, olive oil, cacao oil, apricot-kernel oil, kukui nut oil, sesame oil, wheat germ oil, rice embryo oil, rice bran oil, safflower oil, shea butter, soybean oil, evening primrose oil, perilla oil, tea fruit oil, camellia oil, corn oil, rapeseed oil, hydrogenated rapeseed oil, palm seed oil, hydrogenated palm seed oil, palm oil, hydrogenated palm oil, peanut oil, hydrogenated peanut oil, castor oil, hydrogenated castor oil, sunflower oil, grape seed oil, jojoba oil, hydrogenated jojoba oil, macademia nut oil, meadowfoam oil, cottonseed oil, hydrogenated cottonseed oil, coconut oil and hydrogenated coconut oil and the like.

Moreover, the natural wax include beeswax, high-acid value beeswax, lanoline, reduced lanoline, hydrogenated lanoline, liquid lanoline, carnauba wax, montan wax and white beeswax and the like.

Among these oily components, white petrolatum and squalane are preferable because of their low-irritant properties. Herein, the oily components used in this invention does not include fatty acids having skin-irritant properties and monovalent higher alcohols having six or more carbon atoms which form esters with the active ingredient and cause to reduce the contents of the active ingredient.

One kind or two or more kinds of the above-mentioned oily components can be used, and the contents in the external preparation preferably range from 10 to 40%, and more preferably from 15 to 35%.

On the other hand, the acidic water-soluble polymer used in the present invention includes carboxyvinyl polymer, polyacrylic acid and maleic-acrylic acid copolymer and the like. Among them, carboxyvinyl polymer is preferable.

Among these acidic water-soluble polymers, the commercially available products of carboxyvinyl polymer include Hibiswako (registered trademark, manufactured by Wako Pure Chemical Industries, Ltd.), Synthalen (registered trademark, available from 3V Group) and Carbopol (registered trademark, available from Goodrich Corporation, USA) and the like. The commercially available products of polyacrylic acid include Junlon (registered trademark, Nihon Junyaku KK) and the like. The commercially available products of maleic-acrylic acid copolymer include VEMA (registered trademark, manufactured by Daicel Chemical Industries, Ltd.) and the like.

The contents of the acidic water-soluble polymer in the emulsion-type external preparations range from 0.01 to 10%. In case that the contents are lower than 0.01%, the sufficient velocity may be not attained. In the case that the contents are higher than 10%, the preparation is sticky when used.

In the present invention, the above-mentioned acidic water-soluble polymers are used by treating with the basic substance to neutralize a part or the whole of the acidic group. Such basic substances include, for example, sodium hydroxide, potassium hydroxide, an alkanolamine such as diethanolamine, diisopropanolamine and triethanolamine. Among them, sodium hydroxide can be preferable used.

While the pH value of 1% aqueous solution of carboxyvinyl polymer is 2 to 2.5 (strongly acidic), carboxyvinyl polymer is used as an aqueous solution having the pH value of 6 to 10 by treating with the above-mentioned basic substance in the present invention.

In producing the emulsion-type external preparation of the present invention, it is necessary that the emulsified compositions containing the salt of alkali metal or alkali earth metal of the carboxylic acid-type pharmaceuticals and the hydrates thereof as the active ingredient, and the solution of the acidic water-soluble polymer treated with the basic substance are prepared separately, and then mixed together.

In more detail, the above-mentioned emulsified compositions can be obtained by emulsifying the aqueous phase (component (a)) containing the salt of alkali metal or alkali earth metal of the carboxylic acid-type pharmaceuticals and the hydrates thereof and the aqueous solvent and the oily phase (component (b)) consisting of one kind or two or more kinds of the oily components selected from the group consisting of the ester of fatty acid, hydrocarbon, animal or plant oil or hydrogenated oil thereof and the natural wax.

The component (a) which is the aqueous phase can be prepared by mixing the salt of alkali metal or alkali earth metal of the carboxylic acid-type pharmaceuticals and the hydrates thereof and the aqueous solvent, if necessary under heating at about 55° C. to about 85° C. according to a conventional manner.

The component (b) which is the oily phase can be prepared by mixing the above-mentioned oily components, if desired by adding a surfactant and a preservative and the like thereto among the below-mentioned arbitrary ingredient, if necessary under heating at about 55° C. to about 85° C. according to a conventional manner.

The emulsified compositions can be prepared by emulsifying the aqueous phase and the oily phase obtained according to the above-mentioned manner with a conventional emulsifying procedure such as phase-inversion emulsification method and the like. In more detail, the emulsified compositions can be obtained by adding gradually the oily component while stirring to the aqueous phase, and premixing, and then emulsifying the who mixture with a vacuum emulsifier.

On the other hand, a part or the whole of the acidic water-soluble polymer is treated with the basic substance in the above-mentioned aqueous solvent, converted to the treated acidic water-soluble polymer solution, and then mixed with the above-mentioned emulsified compositions. The acidic water-soluble polymer treated with the basic substance in the aqueous solvent may be the whole or a part of the acidic water-soluble polymer to be added to the external preparations. Herein, in view for simplifying the production process, the whole of the acidic water-soluble polymer is preferably added. In case that a part of the acidic water-soluble polymer is added, the ratio of the treated acidic water-soluble polymer against the untreated acidic water-soluble polymer ranges preferably from 1:0.001 to 2.5, more preferably from 1:0.001 to 1, and particularly preferably from 1:0.001 to 0.3.

The solution containing the treated acidic water-soluble polymer can be obtained by dissolving the acidic water-soluble polymer and the basic substance in the aqueous solvent. The basic substance is preferably used so that the pH value of such solution can be adjusted to 6 to 10.

In the case that a part of the acidic water-soluble polymer is merely treated, the untreated acidic water-soluble polymer is dissolved in the aqueous solvent separately. Such solution is mixed with the above-mentioned aqueous phase and oily phase and the whole mixture is emulsified so that the untreated acidic water-soluble polymer can be dispersed in the emulsified compositions.

According to the above-mentioned manner, the emulsion-type external preparations of the present invention can be obtained by mixing the emulsified compositions obtained by emulsifying the component (a) and the component (b) with the treated acidic water-soluble polymer solution. The mixing procedures can be carried out in a conventional manner, and the emulsified compositions is preferably mixed after cooling at 55° C. or below. The obtained emulsion-type external preparations are O/W type emulsion.

The pH value of the emulsion-type external preparations obtained according to the above manner preferably ranges from 6 to 9, and more preferably from 7 to 9. The range of the pH value can be appropriately adjusted by using the kind of the acidic water-soluble polymer, or contents thereof and the like.

Herein, the pH value was measured by dissolving the sample in 30 mL of water and employing a conventional pH measurement instrument such as Seven Multi S40 (available from Mettler-Toledo Inc).

To the emulsion-type external preparations of the present invention prepared by the above-mentioned manner can be added arbitrary ingredients such as a surfactant, an antioxidant, a preservative, a stabilizer, a solubilizers, a buffer, a flavoring agent, a humectant and the like, if necessary.

The surfactants in the arbitrary ingredients include, for example, a propyleneglycol fatty acid ester, a glycerin fatty acid ester, a polyglycerin fatty acid ester, a sorbitan fatty acid ester, a polyoxyethylene (herein after referred to as POE) sorbitan fatty acid ester, a POE sorbit fatty acid ester, a POE glycerin fatty acid ester, a POE alkyl ether, a POE fatty acid ester, a POE hydrogenated castor oil, a POE castor oil, hydrogenated soybean phospholipid and the like. Herein, examples of fatty acid include lauric acid, myristic acid, palmic acid, stearic acid, linoleic acid and oleic acid. Examples of ester include monoester, diester, triester and sesquiester. Examples of alkyl include lauryl, myristyl, palmityl and stearyl.

The antioxidants include sodium hydrogen sulfite, sodium sulfite, sodium pyrosulfite, parahydroxyanisole, butylhydroxyanisole, dibutylhydroxytoluene, ascorbyl stearate, ascorbyl palmitate, octyl gallate, propyl gallate, tocopherol and the like.

Further, the preservatives include methyl parahydroxybenzoate, propyl parahydroxybenzoate, butyl parahydroxybenzoate and the like.

Furthermore, buffers include sodium citrate, glycine and alanine, the flavoring agents include l-menthol and dl-camphor, and the humectants include sodium hyaluronate, respectively.

The emulsion-type external preparations of the present invention obtained according to the above-mentioned manner can be formulated into a cream or lotion preparation and used as a prescribed external preparation and a quasi drug and the like.

### EXAMPLES

The present invention is illustrated in more detail by the following examples, but these examples are not to be construed to limit the scope of the present invention.

### Example 1

### Preparing Cream Preparation (1)

Cream preparation was prepared according to the following composition and method.

| (Composition) | (Part by weight) |
|---|---|
| (1) Diclofenac sodium | 1 |
| (2) White petrolatum | 10 |
| (3) Cetyl palmitate | 5 |
| (4) Isopropyl myristate | 5 |
| (5) Propylene glycol | 5 |
| (6) Squalane | 5 |
| (7) I-Menthol | 3 |
| (8) Polyoxyl stearate | 2.5 |
| (9) Polyoxyethylene hydrogenated castor oil 60 | 1.5 |
| (10) Methyl parahydroxybenzoate | 0.1 |
| (11) Butyl parahydroxybenzoate | 0.1 |
| (12) Carboxyvinyl polymer*¹ | 0.8 |
| (13) Sodium hydroxide | suitable amount |
| (14) Purified water | q.s. to adjust to |
| | 100 parts |

*¹HIVISWAKO (Manufactured by Wako Pure Chemical Industries, Ltd.)

(Preparing Method)

An oily phase was prepared by heating a mixture of white petrolatum, cetyl palmitate, isopropyl myristate, squalane, polyoxyl stearate, polyoxyethylene hydrogenated castor oil 60, l-menthol and butyl parahydroxybenzoate at 55 to 85° C. to dissolve the whole mixture. An aqueous phase was prepared by mixing 3 parts by weight of propylene glycol and purified water and heating the solution at 55 to 85° C. and adding diclofenac sodium thereto. The aqueous phase and the oily phase were heated at 55° C. to 85° C. and the mixture was emulsified with a vacuum emulsifier to prepare the emulsion composition.

On the other hand, a carboxyvinyl polymer solution was prepared by dissolving the whole amount of carboxyvinyl polymer in purified water to make the concentration of 0.5% to 10% and adding a solution of methyl parahydroxybenzoate in the residual amount of propylene glycol thereto. Sodium hydroxide was added to the obtained carboxyvinyl polymer solution to obtain carboxyvinyl polymer-containing solutions having pH values of 6 to 9. The addition amount of sodium hydroxide was such that the pH values of the finally prepared cream preparations were adjusted to 6, 7, 8, 8.2, 8.3, 8.4, 8.5, 8.6, 8.8 and 9. The carboxyvinyl polymer-containing solutions and the emulsified composition which was cooled to 55° C. or below were mixed and cooled to obtain the 0/W type cream preparations.

### Example 2

### Preparing Cream Preparation (2)

Cream preparation was prepared according to composition of Example 1 and the following method.

(Preparing Method)

An oily phase was prepared by heating a mixture of white petrolatum, cetyl palmitate, isopropyl myristate, squalane, polyoxyl stearate, polyoxyethylene hydrogenated castor oil 60, l-menthol and butyl parahydroxybenzoate at 55° C. to 85° C. to dissolve the whole mixture. An aqueous phase was prepared by mixing 3 parts by weight of propylene glycol and purified water and heating the solution at 55 to 85° C. and adding diclofenac sodium thereto. A solution was prepared by dissolving 75% of a total carboxyvinyl polymer in purified water to make the concentration of polymer to 0.5 to 10%. The solution, the aqueous phase and the oily phase were mixed and emulsified at 55° C. to 85° C. to prepare the emulsified composition in a similar manner as in Example 1.

The residual amount of carboxyvinyl polymer solution was prepared by dissolving the whole amount of carboxyvinyl polymer in purified water to make the concentration of polymer to 0.5 to 10% and adding a solution of methyl parahydroxybenzoate in the residual amount of propylene glycol thereto. Sodium hydroxide was added to the obtained carboxyvinyl polymer solution in order to obtain carboxyvinyl polymer-containing solutions having pH values of 6 to 9. The addition amount of sodium hydroxide was such that the pH values of the finally prepared cream preparations were adjusted to 6, 7, 8, 8.3, 8.4, 8.5, 8.6, 8.8 and 9. The carboxyvinyl polymer-containing solutions and the emulsified composition which was cooled to 55° C. or below were mixed and cooled to obtain the 0/W type cream preparations.

### Example 3

### Preparing Cream Preparation (3)

The O/W type cream preparations were prepared according to the composition of Example 1 and the method of Example 2 except that 50% of total carboxyvinyl polymer was dissolved in purified water to make the concentration of polymer to 0.5 to 10% to prepare a solution, and then the solution was mixed with the aqueous phase and the oily phase and emulsified to prepare the emulsified composition.

### Example 4

### Preparing Cream Preparation (4)

The O/W type cream preparations were prepared according to the composition of Example 1 and the method of Example 2 except that 25% of total carboxyvinyl polymer was dissolved in purified water to make the concentration of polymer to 0.5 to 10% to prepare a solution, and then the solution was mixed with the aqueous phase and the oily phase and emulsified to prepare the emulsified composition.

### Example 5

### Preparing Cream Preparation (5)

Cream preparation was prepared according to the following compositions and methods.

| (Composition) | (Part by weight) |
|---|---|
| (1) Diclofenac sodium | 0.5 |
| (2) White petrolatum | 5 |
| (3) Cetyl palmitate | 5 |
| (4) Myristyl myristate | 20 |
| (5) Glycerol | 5 |
| (6) Liquid paraffin | 5 |
| (7) Stearyl stearate | 2.5 |
| (8) Hydrogenated castor oil | 1.5 |
| (9) Methyl parahydroxybenzoate | 0.1 |
| (10) Propyl parahydroxybenzoate | 0.1 |
| (11) Carboxyvinyl polymer*¹ | 0.1 |
| (12) Potassium hydroxide | suitable amount |
| (13) Purified water | q.s. to adjust to 100 parts |

(Preparing Method) An oily phase was prepared by heating a mixture of white petrolatum, cetyl palmitate, myristyl myristate, liquid paraffin, stearyl stearate, hydrogenated castor oil and propyl parahydroxybenzoate at 55 to 85° C. to dissolve the whole mixture. An aqueous phase was prepared by mixing 3 parts by weight of glycerol and purified water and heating the solution at 55 to 85° C. and adding diclofenac sodium thereto. The aqueous phase and the oily phase were emulsified at 55 to 85° C., to prepare the emulsified composition in a similar manner as in Example 1.

On the other hand, a carboxyvinyl polymer solution was prepared by dissolving carboxyvinyl polymer in purified water to make the concentration of polymer to 0.5 to 10% and adding a solution of methyl parahydroxybenzoate in the residual amount of glycerol thereto. Potassium hydroxide was added to the obtained carboxyvinyl polymer solution in order to obtain carboxyvinyl polymer-containing solutions having pH values of 6 to 9. The addition amount of potassium hydroxide was such that the pH values of the finally prepared cream preparations were adjusted to 6, 7, 8, 8.3, 8.4, 8.5, 8.6, 8.8 and 9. The carboxyvinyl polymer-containing solutions and the emulsion composition which was cooled to 55° C. or below were mixed and cooled to obtain the O/W type cream preparations.

### Example 6

### Preparing Cream Preparation (6)

Cream preparation was prepared according to the following composition and method.

| (Composition) | (Part by weight) |
|---|---|
| (1) Diclofenac sodium | 5 |
| (2) Solid paraffin | 15 |
| (3) Beeswax | 5 |
| (4) Octyldodecyl oleate | 20 |
| (5) 1,3-Butylene glycol | 5 |
| (6) Liquid paraffin | 10 |
| (7) Glyceryl monostearate | 0.5 |
| (8) Sucrose stearate | 3.0 |
| (9) Methyl parahydroxybenzoate | 0.1 |
| (10) Butyl parahydroxybenzoate | 0.1 |
| (11) Carboxyvinyl polymer*¹ | 1.0 |
| (12) Diethanolamine | suitable amount |
| (13) Purified water | q.s. to adjust to 100 parts |

An oily phase was prepared by heating a mixture of solid paraffin, beeswax, octyldodecyl oleate, liquid paraffin, glyceryl monostearate, sucrose stearate and butyl parahydroxybenzoate at 55 to 85° C. to dissolve the who mixture. An aqueous phase was prepared by mixing 2 parts by weight of 1,3-butylene glycol and purified water and heating the solution at 55 to 85° C. and adding diclofenac sodium thereto. A solution was prepared by dissolving 25% of a total carboxyvinyl polymer in purified water to make the concentration of polymer to 0.5 to 10%. The obtained solution, the aqueous phase and the oily phase were mixed and emulsified at 55 to 85° C. and prepare the emulsified composition in a similar manner as in Example 1.

The residual amount of carboxyvinyl polymer solution was prepared by dissolving the whole amount of carboxyvinyl polymer in purified water to make the concentration of polymer to 0.5 to 10% and adding a solution of methyl parahydroxybenzoate in the residual amount of 1,3-butylene glycol thereto. Diisopropanolamine was added to the obtained carboxyvinyl polymer solution to obtain carboxyvinyl polymer-containing solutions having pH values of 6 to 9. The addition amount of diisopropanolamine was such that the pH values of the finally prepared cream preparations were adjusted to 6, 7, 8, 8.3, 8.4, 8.5, 8.6, 8.8 and 9. The carboxyvinyl polymer-containing solutions and the emulsified composition which was cooled to 55° C. or below were mixed and cooled to obtain the 0/W type cream preparations.

### Example 7

### Preparing Cream Preparation (7)

Cream preparation was prepared according to the following composition and method.

| (Composition) | (Part by weight) |
|---|---|
| (1) Loxoprofen sodium | 3.5 |
| (2) Seresin | 12 |
| (3) Shellac | 3 |
| (4) Isopropyl palmitate | 8 |
| (5) Polyethylene glycol 300 | 10 |
| (6) Squalane | 5 |
| (7) Polyoxyethylene monostearyl ether 20 | 2.4 |
| (8) Polyoxyethylene monolaurate sorbitan 15 | 1.6 |
| (9) Methyl parahydroxybenzoate | 0.1 |
| (10) Dibutylhydroxytoluene | 0.5 |
| (11) Carboxyvinyl polymer*¹ | 1.0 |
| (12) Sodium hydroxide | suitable amount |
| (13) Purified water | q.s. to adjust to 100 parts |

(Preparing Method)

An oily phase was prepared by heating a mixture of seresin, shellac, isopropyl palmitate, squalane, polyoxyethylene monostearyl ether 20, polyoxyethylene monolaurate sorbitan 15 and dibutylhydroxytoluene at 55 to 85° C. to dissolve the whole mixture. An aqueous phase was prepared by mixing 2 parts by weight of polyethylene glycol 300 and purified water and heating the solution at 55 to 85° C. and adding loxoprofen sodium thereto. A solution was prepared by dissolving 25% of a total carboxyvinyl polymer in purified water to make the concentration of polymer to 0.5 to 10%. The obtained solution, the aqueous phase and the oily phase were mixed and emulsified at 55 to 85° C. to prepare the emulsified composition in a similar manner as in Example 1.

The residual amount of carboxyvinyl polymer solution was prepared by dissolving the whole amount of carboxyvinyl polymer in purified water to make the concentration of polymer to 0.5 to 10% and adding a solution of methyl parahydroxybenzoate in the residual amount of polyethylene glycol 300 thereto. Sodium hydroxide was added to the obtained carboxyvinyl polymer solution to obtain carboxyvinyl polymer-containing solutions having pH values of 6 to 9. The addition amount of sodium hydroxide was such that the pH values of the finally prepared cream preparations were adjusted to 6, 7, 8, 8.3, 8.4, 8.5, 8.6, 8.8 and 9. The carboxyvinyl polymer-containing solutions and the emulsion composition which was cooled to 55° C. or below were mixed and cooled to obtain the O/W type cream preparations.

### Example 8

### Preparing Cream Preparation (8)

Cream preparation was prepared according to the following composition and method.

| (Composition) | (Part by weight) |
|---|---|
| (1) Loxoprofen sodium | 0.1 |
| (2) White petrolatum | 8 |
| (3) Lanoline | 3 |
| (4) Octyldodecyl myristate | 5 |
| (5) Propylene glycol | 5 |
| (6) Squalane | 5 |
| (7) Polyoxyl stearate | 2.5 |
| (8) Polyoxyethylene hydrogenated castor oil 50 | 2.5 |
| (9) Methyl parahydroxybenzoate | 0.1 |
| (10) Propyl parahydroxybenzoate | 0.1 |
| (11) Dibutylhydroxyanisole | 0.1 |
| (11) Polyacrylic acid*² | 2.5 |
| (12) Potassium hydroxide | suitable amount |
| (13) Purified water | q.s. to adjust to |
| | 100 parts |

*²JYUNRON (Manufactured by Nippon Pure Chemical Co., Ltd.)

(Preparing Method)

An oily phase was prepared by heating a mixture of white petrolatum, lanoline, octyldodecyl myristate, squalane, polyoxyl stearate, dibutylhydroxyanisole, polyoxyethylene hydrogenated castor oil 50 and propyl parahydroxybenzoate at 55 to 85° C. to dissolve the whole mixture. An aqueous phase was prepared by mixing 3 parts by weight of propylene glycol and purified water and heating the solution at 55 to 85° C. and adding loxoprofen sodium thereto. The aqueous phase and the oily phase were emulsified at 55 to 85° C. to prepare the emulsified composition in a similar manner as in Example 1.

On the other hand, a polyacrylic acid solution was prepared by dissolving polyacrylic acid in purified water to make the concentration of polymer to 0.5 to 10% and adding a solution of methyl parahydroxybenzoate in the residual amount of propylene glycol thereto. Potassium hydroxide was added to the obtained polyacrylic acid solution to obtain polyacrylic acid-containing solutions having pH values of 6 to 9. The addition amount of potassium hydroxide was such that the pH values of the finally prepared cream preparations were adjusted to 6, 7, 8, 8.3, 8.4, 8.5, 8.6, 8.8 and 9. The polyacrylic acid-containing solutions and the emulsified composition which was cooled to 55° C. or below were mixed and cooled to obtain the O/W type cream preparations.

### Example 9

### Preparing Cream Preparation (9)

Cream preparation was prepared according to the following composition and method.

| (Composition) | (Part by weight) |
|---|---|
| (1) Loxoprofen sodium | 5.0 |
| (2) White petrolatum | 8 |
| (3) Cetyl palmitate | 1 |
| (4) Isopropyl myristate | 5 |
| (5) Propylene glycol | 5 |
| (6) Liquid paraffin | 5 |
| (7) Polyoxyethylene glycerin stearate | 1.5 |
| (8) Polyoxyethylene hydrogenated castor oil 50 | 2.5 |
| (9) Methyl parahydroxybenzoate | 0.1 |
| (10) Propyl parahydroxybenzoate | 0.1 |
| (11) Sodium pyrosulfite | 0.1 |
| (11) Maleic anhydride-methoxyethylene copolymer*³ | 2.5 |
| (12) Potassium hydroxide | suitable amount |
| (13) Purified water | q.s. to adjust to |
| | 100 parts |

*³VEMA (Manufactured by Daicel Chemical Industries, Ltd.)

(Preparing Method)

An oily phase was prepared by heating a mixture of white petrolatum, cetyl palmitate, isopropyl myristate, liquid paraffin, polyoxyethylene glycerin stearate, polyoxyethylene hydrogenated castor oil 50 and propyl parahydroxybenzoate at 55 to 85° C. to dissolve the whole mixture. An aqueous phase was prepared by mixing 3 parts by weight of propylene glycol and purified water and heating the solution at 55 to 85° C. and adding loxoprofen sodium thereto. The aqueous phase and the oily phase were emulsified at 55 to 85° C. to prepare the emulsified composition in a similar manner as in Example 1.

On the other hand, a maleic anhydride-methoxyethylene copolymer solution was prepared by dissolving maleic anhydride-methoxyethylene copolymer in purified water to make the concentration of 0.5 to 10% and adding a solution of methyl parahydroxybenzoate and sodium pyrosulfite in the residual amount of propylene glycol thereto. Potassium hydroxide was added to polyacrylic acid in order to adjust the pH values of the finally prepared cream preparations to 6, 7, 8, 8.3, 8.4, 8.5, 8.6, 8.8 and 9 to obtain polyacrylic acid-containing solutions. The pH values of these solutions were 6 to 9. The polyacrylic acid-containing solutions and the emulsified composition which was cooled to 55° C. or below were mixed and cooled to obtain the 0/W type cream preparations.

### Examples 10 to 18

### Preparing Lotions (1) to (9)

The lotion preparations were prepared according to the formulations of the cream preparations described in Example 1 to Example 9 except that in each method, a half amount of the acidic water-soluble polymer (carboxyvinyl polymer, polyacrylic acid, maleic anhydride-methoxyethylene copolymer) was used in each composition.

### Example 19

### Preparing Cream Preparation (10)

Cream preparation was prepared according to the following composition and method.

| (Composition) | (Part by weight) |
|---|---|
| (1) Sodium cromoglicate | 1.0 |
| (2) White petrolatum | 8 |
| (3) Cetyl palmitate | 1 |
| (4) Octyldodecyl myristate | 3 |
| (5) 1,3-Butylene glycol | 5 |
| (6) Liquid paraffin | 5 |
| (7) Polyoxyethylene glycerin stearate | 1.5 |
| (8) Polyoxyethylene hydrogenated castor oil 50 | 1.5 |
| (9) Methyl parahydroxybenzoate | 0.1 |
| (10) Propyl parahydroxybenzoate | 0.1 |
| (11) Sodium pyrosulfite | 0.1 |
| (11) Carboxyvinyl polymer*⁴ | 0.7 |
| (12) Triethanolamine | suitable amount |
| (13) Purified water | q.s. to adjust to |
| | 100 parts |

*⁴SYNTHALEN (Manufactured by 3V Group)

(Preparing Method)

An oily phase was prepared by heating a mixture of white petrolatum, cetyl palmitate, octyldodecyl myristate, liquid paraffin, polyoxyethylene glycerin stearate, polyoxyethylene hydrogenated castor oil 50 and propyl parahydroxybenzoate at 55 to 85° C. to dissolve the whole mixture. An aqueous phase was prepared by mixing 3 parts by weight of 1,3-butylene glycol and purified water and heating the solution at 55 to 85° C. and adding sodium cromoglicate thereto. The aqueous phase and the oily phase were emulsified at 55 to 85° C. in a similar manner as in Example 1 to prepare the emulsified composition. A carboxyvinyl polymer solution was prepared by dissolving carboxyvinyl polymer in purified water to make the concentration of polymer to 0.5 to 10% and adding a solution of methyl parahydroxybenzoate and sodium pyrosulfite in the residual amount of 1,3-butylene glycol thereto. Triethanolamine was added to the obtained carboxyvinyl polymer to obtain carboxyvinyl polymer-containing solutions having pH values of 6 to 9. The addition amount of triethanolamine was such that the pH values of the finally prepared cream preparations were adjusted to 6, 7, 8, 8.3, 8.4, 8.5, 8.6, 8.8 and 9. The carboxyvinyl polymer-containing solutions and the emulsified composition which was cooled to 55° C. or below were mixed and cooled to obtain the O/W type cream preparations.

### Example 20

### Preparing Cream Preparation (11)

Cream preparation was prepared according to the following compositions and methods.

| (Composition) | part by weight) |
|---|---|
| (1) Pravastatin sodium | 0.5 |
| (2) Beeswax | 12 |
| (3) Hydrogenated castor oil | 1 |
| (4) Octyldodecyl myristate | 3 |
| (5) 1,3-Butylene glycol | 5 |
| (6) Palm oil | 5 |
| (7) Polyoxyethylene glycerin stearate | 1.5 |
| (8) Sorbitan sesquioleate | 2.0 |
| (9) Methyl parahydroxybenzoate | 0.1 |
| (10) Butyl parahydroxybenzoate | 0.1 |
| (11) Sodium pyrosulfite | 0.1 |
| (11) Carboxyvinyl polymer*⁴ | 0.5 |
| (12) Diisopropanolamine | suitable amount |
| (13) Purified water | q.s. to adjust to 100 parts |

(Preparing Method)

An oily phase was prepared by heating a mixture of beeswax, hydrogenated castor oil, octyldodecyl myristate, palm oil, polyoxyethylene glycerin stearate, sorbitan sesquioleate and butyl parahydroxybenzoate at 55 to to dissolve the whole mixture. An aqueous phase was prepared by mixing 3 parts by weight of 1,3-butylene glycol and purified water and heating the solution at 55 to 85° C. and adding pravastatin sodium thereto. The aqueous phase and the oily phase were emulsified at 55 to 85° C. to prepare the emulsified composition in a similar manner as in Example 1. A carboxyvinyl polymer solution was prepared by dissolving carboxyvinyl polymer in purified water to make the concentration of polymer to 0.5 to 10% and adding a solution of methyl parahydroxybenzoate and sodium pyrosulfite in the residual amount of 1,3-butylene glycol thereto. Diisopropanolamine was added to the obtained carboxyvinyl polymer solution to obtain carboxyvinyl polymer-containing solutions having pH values of 6 to 9. The addition amount of diisopropanolamine was such that the pH values of the finally prepared cream preparations were adjusted to 6, 7, 8, 8.3, 8.4, 8.5, 8.6, 8.8 and 9. The carboxyvinyl polymer-containing solutions and the emulsified composition which was cooled to 55° C. or below were mixed and cooled to obtain the 0/W type cream preparations.

### Example 21

### Preparing Cream Preparation (12)

Cream preparation was prepared according to the following composition and method.

| (Composition) | part by weight) |
|---|---|
| (1) Mupirocin calcium hydrate | 3 |
| (2) Solid paraffin | 10 |
| (3) Beeswax | 5 |
| (4) Isopropyl palmitate | 10 |
| (5) 1,3-Butylene glycol | 5 |
| (6) Light liquid paraffin | 10 |
| (7) Glyceryl monostearate | 0.5 |
| (8) Sucrose stearate | 3.0 |
| (9) Methyl parahydroxybenzoate | 0.1 |
| (10) Butyl parahydroxybenzoate | 0.1 |
| (11) Carboxyvinyl polymer*⁴ | 1.0 |
| (12) Diisopropanolamine | suitable amount |
| (13) Purified water | q.s. to adjust to 100 parts |

(Preparing Method)

An oily phase was prepared by heating a mixture of sol id paraffin, beeswax, isopropyl palmitate, light liquid paraffin, glyceryl monostearate, sucrose stearate, and butyl parahydroxybenzoate at 55 to 85° C. to dissolve the who mixture. An aqueous phase was prepared by mixing 2 parts by weight of 1,3-butylene glycol and purified water and heating the solution at 55 to 85° C. and adding mupirocin calcium hydrate thereto. A solution was prepared by dissolving 25% of a total carboxyvinyl polymer in purified water to make the concentration of 0.5 to 10%. The solution, the aqueous phase and the oily phase were mixed and emulsified at 55 to 85° C. to prepare the emulsified composition. A carboxyvinyl polymer solution was prepared by dissolving the residual carboxyvinyl polymer in purified water to make the concentration of polymer to 0.5 to 10% and adding a solution of methyl parahydroxybenzoate in the residual amount of 1,3-butylene glycol thereto. Diisopropanol amine was added to carboxyvinyl polymer in order to adjust the pH values of the finally prepared cream preparations to 6, 7, 8, 8.3, 8.4, 8.5, 8.6, 8.8 and 9 to obtain carboxyvinyl polymer-containing solutions. The pH values of these solutions were 6 to 9. The carboxyvinyl polymer-containing solutions and the emulsified composition which was cooled to 55° C. or below were mixed and cooled to obtain the O/W type cream preparations.

### Comparative Example 1

Cream preparation was prepared according to the following composition and method.

| (Composition) | (Part by weight) |
|---|---|
| (1) Diclofenac sodium | 1 |
| (2) White petrolatum | 10 |
| (3) Cetyl palmitate | 5 |
| (4) Isopropyl myristate | 5 |
| (5) Propylene glycol | 5 |
| (6) Squalane | 5 |
| (7) I-Menthol | 3 |
| (8) Polyoxyl stearate | 2.5 |
| (9) Polyoxyethylene hydrogenated castor oil 60 | 1.5 |
| (10) Methyl parahydroxybenzoate | 0.1 |
| (11) Butyl parahydroxybenzoate | 0.1 |
| (12) Carboxyvinyl polymer*¹ | 0.8 |
| (13) Sodium hydroxide | suitable amount |
| (14) Purified water | q.s. to adjust to |
| | 100 parts |

(Preparing Method)

An oily phase was prepared by heating a mixture of white petrolatum, cetyl palmitate, isopropyl myristate, squalane, polyoxyl stearate, polyoxyethylene hydrogenated castor oil 60, l-menthol and butyl parahydroxybenzoate at 55 to 85° C. to dissolve the whole mixture. An aqueous phase was prepared by mixing diclofenac sodium, methyl parahydroxybenzoate, propylene glycol and purified water, and dissolved by heating at 55 to 85° C. The aqueous phase and the oily phase were emulsified to prepare the emulsified composition in a similar manner as Example 1.

On the other hand, the solution was prepared by dissolving carboxyvinyl polymer in purified water to make the concentration of 0.5 to 10%, and emulsified composition were mixed. Sodium hydroxide was added to the solution in order to adjust the pH values of the finally prepared cream preparations to 8, 8.3, 8.4, 8.5, 8.6, 8.8 and 9 and mixed. The who mixture was cooled to obtain the O/W type cream preparations.

### Comparative Example 2

Cream preparation was prepared according to the following compositions and methods.

| (Composition) | (Part by weight) |
|---|---|
| (1) Diclofenac sodium | 3 |
| (2) White petrolatum | 10 |
| (3) Stearyl alcohol | 10 |
| (4) Isopropyl myristate | 5 |
| (5) Propylene glycol | 5 |
| (6) Squalane | 5 |
| (7) Polyoxyl stearate | 2.5 |
| (8) Polyoxyethylene hydrogenated castor oil 60 | 1.5 |
| (9) Methyl parahydroxybenzoate | 0.1 |
| (10) Butyl parahydroxybenzoate | 0.1 |
| (11) Carboxyvinyl polymer*¹ | 0.8 |
| (12) Sodium hydroxide | suitable amount |
| (13) Purified water | q.s. to adjust to |
| | 100 parts |

(Preparing Method)

An oily phase was prepared by heating a mixture of white petrolatum, stearyl alcohol, isopropyl myristate, squalane, polyoxyl stearate, polyoxyethylene hydrogenated castor oil 60 and butyl parahydroxybenzoate at 55 to 85° C. to dissolve the whole mixture. An aqueous phase was prepared by mixing diclofenac sodium, methyl parahydroxybenzoate, propylene glycol and purified water, and heating the solution at 55 to 85° C. The aqueous phase and the oily phase were emulsified at 55 to 85° C. to prepare the emulsified composition in a similar manner as in Example 1.

On the other hand, the solution was prepared by dissolving carboxyvinyl polymer in purified water to make the concentration of polymer to 0.5 to 10%, were mixed the emulsion composition, and sodium hydroxide was added to the solution in order to adjust the pH values of the finally prepared cream preparations to 6, 7, 8, 8.3, 8.4, 8.5, 8.6, 8.8 and 9, and mixed. The whole mixture was cooled to obtain the O/W type cream preparations.

### Test Example 1

### Stability Test (1)

The cream preparations having the pH values shown in Table 1 which were obtained in accordance with the procedures of Examples 1 to 4 and Comparative Example 1 were stored at −2° C. for three months and crystallization was observed at the beginning of the test and after one week, two weeks, four weeks, one month and three months from the beginning of the test. The results are summarized in Table 1. Herein, the pH values were determined as follows: a solution of 1 g of cream preparation was dissolved in 30 mL of water and pH value of the solution was measured by employing Seven Multi S40 (available from Mettler-Toledo Inc.).

TABLE 1

| | | starting | in 1 | in 2 | in 4 | in 1 | in 3 |
|---|---|---|---|---|---|---|---|
| PH | Creams | time | week | weeks | weeks | month | months |
| 8 | Comparative | X | X | X | X | X | X |
| | example 1 | | | | | | |
| 8.2 | example 1 | ◯ | ◯ | ◯ | ◯ | ◯ | ◯ |
| | Comparative | X | X | X | X | X | X |
| | example 1 | | | | | | |
| 8.3 | example 1 | ◯ | ◯ | ◯ | ◯ | ◯ | ◯ |
| | Comparative | ◯ | ◯ | X | X | X | X |
| | example 1 | | | | | | |
| 8.4 | example 1 | ◯ | ◯ | ◯ | ◯ | ◯ | ◯ |
| | Comparative | ◯ | ◯ | X | X | X | X |
| | example 1 | | | | | | |
| 8.5 | example 1 | ◯ | ◯ | ◯ | ◯ | ◯ | ◯ |
| | example 2 | ◯ | ◯ | ◯ | ◯ | ◯ | ◯ |
| | example 3 | ◯ | ◯ | ◯ | ◯ | ◯ | ◯ |
| | example 4 | ◯ | ◯ | ◯ | ◯ | ◯ | ◯ |
| | Comparative | ◯ | ◯ | X | X | X | X |
| | example 1 | | | | | | |
| 8.6 | example 1 | ◯ | ◯ | ◯ | ◯ | ◯ | ◯ |
| | Comparative | ◯ | ◯ | X | X | X | X |
| | example 1 | | | | | | |
| 8.8 | Comparative | ◯ | X | X | X | X | X |
| | example 1 | | | | | | |
| 9 | Comparative | ◯ | X | X | X | X | X |
| | example 1 | | | | | | |

In Table 1,
Symbol “X” means crystallization,, and
Symbol “◯” means no crystallization.

### Test Example 2

### Stability Test (2)

The cream preparations having the pH values shown in Table 2 which were obtained in accordance with the procedure of Examples 1 to 4 and Comparative Example 1 were stored at 4° C. for twelve months and crystallization was observed at the beginning of the test and after one week, two weeks, four weeks, one month, three months, six months and twelve months from the beginning of the test. The results are summarized in Table 2.

TABLE 2

| | | start- | | | | | |
|---|---|---|---|---|---|---|---|
| | | ing | in 2 | in 1 | in 3 | in 6 | in 12 |
| PH | Creams | time | weeks | month | months | months | months |
| 8 | Comparative | X | X | X | X | X | X |
| | example 1 | | | | | | |
| 8.2 | example 1 | ◯ | ◯ | ◯ | ◯ | ◯ | ◯ |
| | Comparative | X | X | X | X | X | X |
| | example 1 | | | | | | |
| 8.3 | example 1 | ◯ | ◯ | ◯ | ◯ | ◯ | ◯ |
| | Comparative | ◯ | ◯ | ◯ | ◯ | ◯ | X |
| | example 1 | | | | | | |
| 8.4 | example 1 | ◯ | ◯ | ◯ | ◯ | ◯ | ◯ |
| | Comparative | ◯ | ◯ | ◯ | ◯ | ◯ | X |
| | example 1 | | | | | | |
| 8.5 | example 1 | ◯ | ◯ | ◯ | ◯ | ◯ | ◯ |
| | example 2 | ◯ | ◯ | ◯ | ◯ | ◯ | ◯ |
| | example 3 | ◯ | ◯ | ◯ | ◯ | ◯ | ◯ |
| | example 4 | ◯ | ◯ | ◯ | ◯ | ◯ | ◯ |
| | Comparative | ◯ | ◯ | ◯ | ◯ | ◯ | X |
| | example 1 | | | | | | |
| 8.6 | example 1 | ◯ | ◯ | ◯ | ◯ | ◯ | ◯ |
| | Comparative | ◯ | ◯ | ◯ | ◯ | ◯ | X |
| | example 1 | | | | | | |
| 8.8 | Comparative | ◯ | ◯ | ◯ | X | X | X |
| | example 1 | | | | | | |
| 9 | Comparative | ◯ | ◯ | X | X | X | X |
| | example 1 | | | | | | |

In Table 2,
Symbol “X” means crystallization, and
Symbol “◯” means no crystallization.

### Test Example 3

### Stability Test (3)

The cream preparations having the pH values shown in Table 3 which were obtained in accordance with the procedures of Examples 1 to 4 and Comparative Examples 1 and 2 were stored at 40° C. for three months and the contents of diclofenac sodium in the external preparations were measured at the beginning of the test and after the test with HPLC. The residual rate of diclofenac sodium was determined as the rate (%) of the contents of diclofenac sodium after the test based on the contents of diclofenac sodium in the external preparation at the beginning of the test. Further, the formation of an ester from diclofenac and stearyl alcohol was examined with HPLC under the condition as mentioned below. The results are summarized in Table 3.

TABLE 3

| PH | Creams | Residual ratio of diclofenac sodium(%) |
|---|---|---|
| 8 | Comparative example 1 | 94.7 |
| 8.3 | example 1 | 99.7 |
| | Comparative example 1 | 95.8 |
| | Comparative example 2 | 96.8*⁵ |
| 8.4 | Comparative example 1 | 96.2 |
| 8.5 | example 1 | 100.7 |
| | example 2 | 100.8 |
| | example 3 | 100.8 |
| | example 4 | 100.1 |
| | Comparative example 1 | 98.7 |
| | Comparative example 2 | 97.0*⁵ |
| 8.6 | example 1 | 100.9 |
| 8.8 | Comparative example 1 | 97.9 |
| 9 | Comparative example 1 | 96.2 |

*⁵Ester formed diclofenac and stearyl alcohol was detected.

(Measurement of the Contents of Diclofenac Sodium)

The condition of HPLC:
Column: Mightysil RP-18 (Kanto Kagaku)
Mobile phase: 0.12% acetic acid/methanol (1:2)
Volume rate of flow: 1.0 ml/min.
Wavelength for detection: 282 nm

(Measurement of the Formation of Ester of Diclofenac and Stearyl Alcohol)

The condition of HPLC:
Column: Symmetry Shield RP-18 (Nihon Waters K. K.)
Mobile phase A: 0.1% acetic acid
Mobile phase B: Solution of 0.1% acetic acid in methanol
Volume rate of flow: 1.0 ml/min.
Wavelength for detection: 282 nm

<Gradient Condition>

| Time | Mobile phase A | Mobile phase B |
|---|---|---|
| 0 to 11 minutes | 40 to 28% | 60 to 72% |
| 11 to 35 minutes | 28 to 10% | 72 to 90% |

As can be understood from the results shown in Tables 1 and 2, it was confirmed that the cream preparations of Examples 1 to 4 in which calboxylvinyl polymer is treated with the basic substance before being added exhibit less diclofenac sodium precipitation compared to the cream preparations of Comparative Example 1 in which calboxylvinyl polymer is was not treated with the basic substance before being added. Further, the results shown in Table 3 indicate that the cream preparations of Examples 1 to 4 had higher residue amount of diclofenac sodium after storage compared to the cream preparation of the comparative example. In Comparative Example 2, the cream preparation included higher alcohol, and esters of diclofenac sodium and higher alcohol were detected.

### INDUSTRIAL APPLICABILITY

The emulsion-type external preparations of the present invention having the pH value of 6 to 10 as a solution which is prepared by treating the acidic water-soluble polymer with the basic substance in advance can inhibit the crystallization of the salt of alkali metal or alkali earth metal of carboxylic acid-type pharmaceutical compounds such as non-steroidal anti-inflammatory/analgesic agents and excel in the stability that the contents are not reduced in the storage. Further, the emulsion-type external preparations of the present invention are low skin-irritant and high safety because the preparations do not contain a lower alcohol, a fatty acid and the like. Furthermore, because the preparations of the present invention do not contain a higher alcohol, an ester of the active ingredient derived therefrom is not formed and the contents of the active ingredient are not reduced.

Consequently, the present invention can be advantageously applied to the various pharmaceutical fields as the emulsion-type external preparations which contain the salt of alkali metal or alkali earth metal of carboxylic acid-type pharmaceutical compounds such as non-steroidal anti-inflammatory/analgesic agents and the production methods thereof.

1. An O/W type-emulsion-type external preparation having the pH value of 6 to 9 prepared by:
emulsifying the following components (a) and (b):
(a) an aqueous phase containing a pharmaceutical active ingredient compound selected from the group consisting of diclofenac sodium and loxoprofen sodium and an aqueous solvent; and
(b) an oily phase consisting of one or two or more oily components selected from the group consisting of a fatty acid ester, a hydrocarbon, animal and plant oil and hydrogenated oil thereof and a natural wax; and
mixing the emulsion with a treated acidic water-soluble polymer solution obtained by treating the whole or a part of an acidic water-soluble polymer with a basic substance in an aqueous solvent.

2. The emulsion-type external preparation of claim 1, wherein the aqueous solvent of the component (a) is one or two or more solvents selected from the group consisting of water, a glycol and polyvalent alcohol.

3. The emulsion-type external preparation of claim 1, wherein the content of the pharmaceutical active ingredient compound selected from the group consisting of diclofenac sodium and loxoprofen sodium of the component (a) ranges from 0.01 to 10% by weight.

4. The emulsion-type external preparation of claim 1, wherein the content of the pharmaceutical active ingredient compound selected from the group consisting of diclofenac sodium and loxoprofen sodium of the component (a) ranges from 0.5 to 5% by weight.

5. The emulsion-type external preparation of claim 1, wherein the acidic water-soluble polymer is carboxyvinyl polymer.

6. The emulsion-type external preparation of claim 1, wherein the basic substance for treating the acidic water-soluble polymer is a compound selected from the group consisting of sodium hydroxide, potassium hydroxide and an alkanolamine.

7. The emulsion-type external preparation of claim 1, wherein the weight ratio of the acidic water-soluble polymer treated with the basic substance to untreated acidic water-soluble polymer ranges from 1:0.001 to 2.5.

8. The emulsion-type external preparation of claim 1, wherein the weight ratio of the acidic water-soluble polymer treated with the basic substance to untreated acidic water-soluble polymer ranges from 1:0.001 to 1.

9. The emulsion-type external preparation of claim 1, wherein the weight ratio of the acidic water-soluble polymer treated with the basic substance to untreated acidic water-soluble polymer ranges from 1:0.001 to 0.3.

10. The emulsion-type external preparation of claim 1, wherein the pH value of the solution containing acidic water-soluble polymer solution treated with the basic substance ranges from 6 to 10.

11. The emulsion-type external preparation of claim 1, wherein the preparation is a cream or lotion preparation.

12. A method for producing an O/W type-emulsion-type external preparation having the pH value of 6 to 9 comprising:
emulsifying the following components (a) and (b):
(a) an aqueous phase containing a pharmaceutical active ingredient compound selected from the group consisting of diclofenac sodium and loxoprofen sodium and an aqueous solvent: and
(b) an oily phase consisting of one or two or more oily components selected from the group consisting of a fatty acid ester, a hydrocarbon, animal and plant oil and a hydrogenated oil thereof and a natural wax; and
mixing the emulsion with a treated acidic water-soluble polymer solution obtained by treating the whole or a part of an acidic water-soluble polymer with a basic substance in an aqueous solvent.

13. The method for producing the emulsion-type external preparation of claim 12, wherein the aqueous solvent is one or two or more solvents selected from the group consisting of water, a glycol and polyvalent alcohol.

14. The method for producing the emulsion-type external preparation of claim 12, wherein the content of the pharmaceutical active ingredient compound selected from the group consisting of diclofenac sodium and loxoprofen sodium of the component (a) ranges from 0.01 to 10% by weight.

15. The method for producing the emulsion-type external preparation of claim 12, wherein the content of the pharmaceutical active ingredient compound selected from the group consisting of diclofenac sodium and loxoprofen sodium of the component (a) ranges from 0.5 to 5% by weight.

16. The method for producing the emulsion-type external preparation of claim 12, wherein the acidic water-soluble polymer is carboxyvinyl polymer.

17. The method for producing the emulsion-type external preparation of claim 12, wherein the basic substance is a compound selected from the group consisting of sodium hydroxide, potassium hydroxide and an alkanolamine.

18. The method for producing the emulsion-type external preparation of claim 12, wherein the weight ratio of the acidic water-soluble polymer treated with the basic substance to untreated acidic water-soluble polymer ranges from 1:0.001 to 2.5.

19. The method for producing the emulsion-type external preparation of claim 12, wherein the weight ratio of the acidic water-soluble polymer treated with the basic substance to untreated acidic water-soluble polymer ranges from 1:0.001 to 1.

20. The method for producing the emulsion-type external preparation of claim 12, wherein the weight ratio of the acidic water-soluble polymer treated with the basic substance to untreated acidic water-soluble polymer ranges from 1:0.001 to 0.3.

21. The method for producing the emulsion-type external preparation of claim 12, wherein the pH value of the acidic water-soluble polymer solution treated with the basic substance ranges from 6 to 10.

22. The method for producing the emulsion-type external preparation of claim 12, wherein the emulsion-type external preparation is a cream preparation.

23. A method for inhibiting the crystallization of a pharmaceutical active ingredient compound selected from the group consisting of diclofenac sodium and loxoprofen sodium in an O/W type-emulsion-type external preparation having the pH value of 6 to 9, comprising emulsifying the following components (a) and (b):
(a) an aqueous phase containing a pharmaceutical active ingredient compound selected from the group consisting of diclofenac sodium and loxoprofen sodium; and
(b) an oily phase consisting of one kind or two or more oily components selected from the group consisting of a fatty acid ester, hydrocarbon, animal or plant oil and hydrogenated oil thereof and a natural wax; and
mixing the emulsion with an acidic water-soluble polymer solution which is obtained by treating the whole or a part of the acidic water-soluble polymer with a basic substance in an aqueous solvent.

24. The method for inhibiting the crystallization of claim 23 comprising further mixing with a solution containing an acidic water-soluble polymer untreated with a basic substance.

25. The method for inhibiting the crystallization of claim 23, wherein the acidic water-soluble polymer is a member selected from the group consisting of carboxyvinyl polymer, polyacrylic acid and acrylic acid-maleic acid copolymer.

26. The method for inhibiting the crystallization of claim 23, wherein the basic substance is a compound selected from the group consisting of sodium hydroxide, potassium hydroxide and an alkanolamine.

27. The method for inhibiting the crystallization of claim 23 where in the pH value of the solution containing acidic water-soluble polymer treated with the basic substance ranges from 6 to 10.

28. The method for inhibiting the crystallization of claim 23, wherein the emulsion-type external preparation is a cream preparation.
